(19) United States
(12) Patent Application Publication
(10) Pub. No.: US 2010/0240936 A1
(43) Pub. Date: Sep. 23, 2010

(54) CATALYSTS AND PROCESSES FOR SELECTIVE HYDROGENATION OF ACETYLENE AND DIENES IN LIGHT OLEFIN FEEDSTREAMS

(76) Inventors: Yongqing Zhang, Hoboken, NJ (US); Stephen J. Golden, Santa Barbara, CA (US)

Correspondence Address: REED SMITH LLP, 2500 ONE LIBERTY PLACE, 1650 MARKET STREET, PHILADELPHIA, PA, 19103, US

(21) Appl. No.: 12/791,660

(22) Filed: Jun. 1, 2010

Related U.S. Application Data
(63) Continuation of application No. 12/315,493, filed on Dec. 3, 2008, now abandoned.
Application No. 12/315,493 is a continuation of application No. 11/156,981, filed on Jun. 20, 2005, now abandoned.
(60) Provisional application No. 60/582,559, filed on Jun. 23, 2004.
(60) Provisional application No. 60/582,747, filed on Jun. 23, 2004.
(60) Provisional application No. 60/582,568, filed on Jun. 23, 2004.
(60) Provisional application No. 60/582,534, filed on Jun. 23, 2004.

(51) Int. Cl. B01J 21/08; B01J 23/58; B01J 23/72; B01J 27/135; B01J 27/128; B01J 27/13; B01J 31/02; B01J 27/232; B01J 27/25; B01J 23/46; C07C 7/163; B01J 23/42; B01J 23/44; B01J 23/50; B01J 23/52; B01J 21/04; B01J 21/12

(52) U.S. Cl. 585/262; 502/302; 502/328; 502/330; 502/331; 502/226; 502/227; 502/229; 502/230; 502/170; 502/174; 502/201; 502/326; 585/259; 502/242; 502/245; 502/243; 502/250; 502/258; 502/259; 502/260; 502/261; 502/262; 502/327

(57) ABSTRACT

A catalyst and a method for selective hydrogenation of acetylene and dienes in light olefin feedstreams are provided. The catalyst retains higher activity and selectivity after regeneration than conventional selective hydrogenation catalysts. The catalyst contains a first component and a second component supported on an inorganic support. The inorganic support contains at least one salt or oxide of zirconium, a lanthanide, or an alkaline earth.

### RELATED APPLICATIONS

This application claims the benefit under 35 U.S.C. 119(e) of U.S. Patent Provisional Application Ser. No. 60/582,559, filed Jun. 23, 2004, U.S. Provisional Patent Application Ser. No. 60/582,747, filed Jun. 23, 2004, U.S. Provisional Patent Application Ser. No. 60/582,568, filed Jun. 23, 2004, and U.S. Provisional Patent Application Ser. No. 60/582,534, filed Jun. 23, 2004, all of which are incorporated herein by reference in their entirety.

### FIELD OF THE INVENTION

This invention relates to a catalyst and a process for selective hydrogenation of dienes and acetylene in light olefin feedstreams.

### BACKGROUND OF THE INVENTION

Light olefins are important feedstocks for production of polymers and chemicals. Light olefins are generally made through pyrolysis or catalytic cracking of refinery gas, ethane, propane, butane, or similar feedstreams, or by fluid catalytic cracking of crude oil cuts. The olefin feed streams that are produced by these processes contain small quantities of acetylene and dienes.

The acetylene and dienes in the light olefin feedstreams can cause poisoning of the polymerization catalyst or can produce undesired chemical byproducts. The acetylene and dienes are therefore generally removed from the light olefin feedstreams through selective hydrogenation on a catalyst normally comprising a silver component, a palladium component, and a silica or alumina carrier, with or without other promoters. It is normally desirable that the catalyst selectively hydrogenate substantially all of the acetylene and dienes to monoolefins while converting only an insignificant amount of the olefin to paraffin.

The selective hydrogenation catalyst deactivates over time, probably because of the deposition of oligomers on the catalyst. Regenerating the selective hydrogenation catalyst by successively passing steam and air over the catalyst at elevated temperature restores the catalyst activity and selectivity to some extent. The catalyst activity and selectivity of the regenerated selective hydrogenation catalyst are generally less than the activity and selectivity of a fresh selective hydrogenation catalyst. There is a need for a selective hydrogenation catalyst composition that retains more activity and selectivity after regeneration than conventional selective hydrogenation catalyst.

The palladium that is used in conventional selective hydrogenation catalyst is expensive. There is a need for selective hydrogenation catalysts that are less expensive than conventional selective hydrogenation catalysts.

There is also a need for selective hydrogenation catalysts that have higher activity and longer lifetimes than conventional selective hydrogenation catalysts.

### SUMMARY OF THE INVENTION

One aspect of the present invention provides a catalyst for selective hydrogenation of acetylene and dienes in a light olefin feedstream. The catalyst contains a first component selected from the group consisting of copper, gold, silver, and mixtures thereof, a second component selected from the group consisting of nickel, platinum, palladium, iron, cobalt, ruthenium, rhodium, and mixtures thereof, an inorganic support, and at least one inorganic salt or oxide selected from the group consisting of zirconium, a lanthanide, an alkaline earth, and mixtures thereof.

Preferably, the inorganic salt or oxide is added to the support by impregnation, kneading, or milling. In an embodiment, the inorganic salt or oxide, the first component, the second component, and the support may be added in any order, the catalyst may contain at least one fluorite. Preferably, the fluorite is formed after calcination, use, or regeneration of the catalyst.

In one embodiment, the first component contains palladium and the second component contains silver. The inorganic salt may be selected from the group consisting of nitrates, acetates, chlorides, carbonates, and mixtures thereof. A weight percent of the inorganic salt or oxide may be in the range of approximately 0.01% to approximately 50% by weight. Advantageously, the catalyst is a multi-phase catalyst. The multi-phase catalyst may be prepared with a water solution of at least two water-soluble salts selected from the group consisting of copper, gold, silver, nickel, platinum, palladium, iron, cobalt, ruthenium, rhodium, zirconium, a lanthanide, an alkaline earth, and mixtures thereof.

Another aspect of the invention provides a process for selectively hydrogenating acetylene and dienes in a light olefin feedstream. The process includes contacting the feedstream with hydrogen in the presence of a catalyst of the present invention. Preferably, the light olefin feedstream contains at least one olefin having a carbon number between C₂ through C₆. For example, the light olefin feedstream may contain at least one olefin selected from the group consisting of ethylene, propylene, butylene, pentene, and hexene. Preferably, the light olefin feedstream is an ethylene feedstream.

In an embodiment, the contacting is at a temperature between approximately 0° C. and approximately 250° C. Preferably, the contacting is at a pressure of approximately 0.01 bar to approximately 50 bar.

Yet another aspect of the invention involves a method of preparing a multi-phase catalyst for selective hydrogenation of acetylene and diene in a light olefin feedstream. The method includes forming a single aqueous solution of at least two water-soluble salts selected from the group consisting of copper, gold, silver, nickel, platinum, palladium, iron, cobalt, ruthenium, rhodium zirconium, a lanthanide, an alkaline earth, and mixtures thereof. The method also includes contacting the single aqueous solution with an inorganic support selected from the group consisting of silica and alumina, and calcining the inorganic support and the single aqueous solution under a condition to form said multi-phase catalyst, where the multi-phase catalyst contains at least one inorganic salt or oxide selected from the group consisting of zirconium, a lanthanide, and an alkaline earth.

Preferably, the method also includes removing the water from the single aqueous solution before calcining. In an embodiment, removing the water includes drying the single aqueous solution. Preferably, the inorganic support is silica or alumina, and the water-soluble salts are salts selected from the group consisting of nitrates, acetates, oxalates, hydroxides, and carbonates.

### DESCRIPTION

Conventional selective hydrogenation catalysts for selective hydrogenation of acetylene and dienes in light olefin feedstreams lose activity and selectivity when they are regenerated. Thus it is an objective of the present invention to provide a catalyst with an improved activity and selectivity.

Accordingly, one aspect of the present invention provides a selective hydrogenation catalyst comprising a first component and a second component on an inorganic support. The first component may comprise silver, copper, gold, or any mixture of silver, copper and gold. The second component may comprise palladium, nickel, platinum, iron, cobalt, ruthenium, rhodium, or mixtures thereof. The inorganic support may comprise silica or alumina.

In one embodiment, at least a portion of the second component may comprise nickel, iron, cobalt, rhodium, or ruthenium in addition to, or in place of, the palladium that is used as the second component in conventional selective hydrogenation catalysts. Nickel, iron, cobalt, or ruthenium used as the second components may be less expensive than the palladium that is used as the second component in conventional selective hydrogenation catalysts. Nickel, iron, cobalt, ruthenium, and rhodium may be less susceptible to poisoning than palladium. Sulfur, arsenic, and other inorganic materials can poison the catalyst.

It is the discovery of the present invention that modifying the silica or alumina support by adding at least one inorganic salt selected from the group consisting of zirconium, one or more lanthanides, one or more alkaline earth metals, and mixtures thereof will increase the activity and/or the selectivity of the selective hydrogenation catalyst after regeneration of the selective hydrogenation catalyst. In one embodiment, the inorganic salts of the present invention may be present on the catalyst in amounts of approximately 0.01% to approximately 50% by weight, or more preferably from approximately 0.05% to approximately 20% by weight, where the percentages of the inorganic salts are calculated on the basis of the oxides. At least one of the inorganic salts or oxides may be a fluorite or may be converted to a fluorite after calcination, use, or regeneration. The inorganic salts may be in the form of nitrates, acetates, chlorides, carbonates, any other suitable salt, or mixtures thereof.

For the purpose of the present invention, yttrium and lanthanum are considered to be lanthanides. The term lanthanide in this application and the appended claims includes any of the elements lanthanum, cerium, praseodymium, neodymium, promethium, samarium, europium, gadolinium, terbium, dysprosium, holmium, erbium, thulium, ytterbium, lutetium, and yttrium.

The first component, the second component, and the inorganic salts of the present invention may be added to the support by any suitable method, including, but not limited to, impregnating the support with a solution of salt or salts; or kneading or milling the first component, second component, and inorganic salt or salts with the support.

The first component, the second component, and the inorganic salts may be added to the support in any order. The first and second components may be added together or separately. The inorganic salts may be added to the support simultaneously with the first component and/or the second component.

When the inorganic salt or salts are calcined, the inorganic salt or salts may be converted, at least in part, to the oxide form. Similarly, calcining the first and/or the second components may convert the components to oxides. The oxides may be oxides of a single salt, or the oxides may be mixed metal oxides. In some cases, the oxides may form fluorites after calcination. The form of oxide that is formed may depend on the calcination conditions. The activity and/or stability of the catalyst may also depend on the calcination conditions.

The inorganic salt or salts and/or the first and second components may be converted to the corresponding oxide or oxides during use or regeneration.

In another embodiment, an oxide or a mixture of oxides of the first component, the second component, or the inorganic salts may be added directly to the catalyst rather than, or in addition to, adding a salt or a mixture of salts to the support and converting the salt or salts to the oxide. All of the components of the catalyst may be added in any order.

The catalyst of the present invention can be a single-phase catalyst or a multi-phase catalyst. A multi-phase catalyst is a catalyst that contains more than one phase. In an embodiment, the multiple phases are intimately mixed A multi-phase catalyst (MPC) may be prepared by forming a single aqueous solution of water-soluble salts, contacting the aqueous solution with an inorganic support, removing the water, and calcining the support and water-soluble salts to obtain the multi-phase catalyst. Multi-phase catalysts are generally found to have higher activity and stability than single-phase catalysts having the same composition.

Although not wishing to be limited by a theory, it is believed that, when the multi-phase catalyst is formed by calcining the mixture of water-soluble salts, an intimate mixture of the two or more phases of the multi-phase catalyst is formed. It is believed that the intimate mixture of the multiple phases of the multi-phase catalyst inhibits the agglomeration or sintering of the multiple phases when the multi-phase catalyst is exposed to high temperatures.

The water-soluble salts that form the multi-phase catalyst may be at least two water-soluble salts of silver, copper, gold, palladium, nickel, platinum, iron, cobalt, ruthenium, rhodium, zirconium, one or more lanthanides, one or more alkaline earths, or mixtures thereof. The multi-phase catalyst can therefore include the components that stabilize the support in addition to the first component and second component. The multi-phase catalyst contains at least one inorganic salt or oxide selected from the group consisting of zirconium, a lanthanide, an alkaline earth, and any mixture thereof. The at least one inorganic salt or oxide of the multi-phase catalyst of the present invention may or may not be one of the water-soluble salts that form the aqueous solution of water-soluble salts.

Any manner of water-soluble salts may be used to form the aqueous solution of water-soluble salts. Suitable water-soluble salts include, but are not limited to, nitrates, acetates, oxalates, hydroxides, oxides, carbonates, etc.

In an embodiment, the water may be removed from the aqueous solution of water-soluble salts before forming the multi-phase catalyst. The water may be removed through evaporation by heating the solution. Alternatively, the water may be removed by blowing air over the aqueous solution of water-soluble salts.

The water-soluble salts that are used to form the multi-phase catalyst may be precipitated with a precipitating agent. The precipitated water-soluble salts may be calcined to form the multi-phase catalyst.

The precipitating agent may be any suitable precipitating agent. Some suitable precipitating agents include, but are not limited to, alkali hydroxides, ammonium hydroxide, citric acid, and oxalic acid.

The mixture of water-soluble salts or the precipitated water-soluble salts may be dried before calcining.

The multi-phase catalyst may be formed from the dried mixture of water-soluble salts or the dried multi-phase catalyst precursor by heating the mixture of water-soluble salts or the multi-phase catalyst precursor to a temperature sufficiently high to form the desired phase chemistry of the multi-phase catalyst. Although the temperature that is sufficiently high depends on the multi-phase catalyst that is to be formed, the water-soluble salts are generally heated to a temperature of approximately 600° C. to approximately 900° C., more preferably to a temperature of approximately 700° C. to 850° C. to form the multi-phase catalyst.

In accordance with embodiments of the present invention, the mixture of water-soluble salts is heated for approximately 1 to approximately 100 hours, approximately 2 to approximately 50 hours, or approximately 3 to approximately 10 hours to form the multi-phase catalyst, although the time may vary, depending on the formulation of the multi-phase catalyst. Suitable conditions for forming the multi-phase catalyst may be determined by one skilled in the art without undue experimentation in view of the teaching of the present invention.

The catalysts are suitable for selective hydrogenation of alkynes and dienes mixed with light olefins. The term “light olefins”, as used in the context of this application, is to be understood to mean all of the olefins having carbon numbers in the range of C₂ through C₆. The term “light olefins” therefore includes ethylene, propylene, butylenes, pentenes, and hexenes. The terms “butylenes”, “pentenes”, and “hexenes” include all of the isomers of butylene, pentene, and hexene.

The hydrogenation can be carried out in the gas phase, the liquid phase, or as a gas/liquid mixture. The amount of hydrogen used is from approximately 0.8 to approximately 5, preferably from approximately 0.95 to approximately 2 times the amount required for reaction with the dienes and/or the acetylene.

The selective hydrogenation is carried out at a space velocity of from approximately 500 to approximately 10,000 m³/hr at a temperature between approximately 0° C. and approximately 250° C. and at a pressure of approximately 0.01 to approximately 50 bar.

### EXAMPLE 1

Catalyst A is prepared as follows. A silica support is impregnated with an aqueous solution of cerium nitrate, zirconyl acetate and lanthanum nitrate. The impregnated support is dried and then calcined. The calcined support is subsequently impregnated with an aqueous solution containing a water-soluble palladium salt and a water-soluble silver salt. The catalyst is dried and calcined.

### EXAMPLE 2

Catalyst B is prepared in the same manner as Catalyst A, except that the silica support is impregnated with an aqueous solution of strontium nitrate rather than an aqueous solution of cerium nitrate, zirconyl acetate, and lanthanum nitrate.

### EXAMPLE 3

Catalyst C is prepared in the same manner as Catalyst A, except that the silica support is impregnated with an aqueous solution containing only a water-soluble palladium salt and a water-soluble silver salt. The catalyst does not contain zirconium, a lanthanide, or an alkaline earth.

### EXAMPLE 4

Catalyst D is prepared in the same manner as Catalyst A, except that the silica support is impregnated with an aqueous solution containing ferric nitrate in place of the water-soluble palladium salt.

### EXAMPLE 5

Catalyst E is prepared in the same manner as Catalyst A, except that the silica support is impregnated with an aqueous solution containing cobalt nitrate in place of the water-soluble palladium salt.

### EXAMPLE 6

Catalyst F is prepared in the same manner as Catalyst A, except that the silica support is impregnated with an aqueous solution containing ruthenium nitrate in place of the water-soluble palladium salt.

### EXAMPLE 7

Catalyst G is prepared in the same manner as Catalyst A, except that the silica support is impregnated with an aqueous solution containing rhodium nitrate in place of the water-soluble palladium salt.

### EXAMPLE 8

Catalyst H is prepared in the same manner as Catalyst A, except that the silica support is impregnated with an aqueous solution containing cobalt nitrate in addition to the water-soluble palladium salt and the water-soluble silver salt. Catalyst H therefore contains both palladium and cobalt as second components.

### EXAMPLE 9

Catalyst I is prepared in the same manner as Catalyst A, except that aqueous solutions of cerium nitrate, zirconyl acetate, lanthanum nitrate, the water-soluble palladium salt, and the water-soluble silver salt are added separately to the support, and the support and the aqueous solution are calcined after each solution is added.

Catalyst A is found to contain a multi-phase catalyst. Catalyst I is a single phase catalyst.

### Testing

An ethylene feedstream containing about 1% acetylene is contacted with Catalyst A in the presence of hydrogen at a pressure of 10 bar at temperatures between approximately 45 and 120° C. The catalyst selectively hydrogenates the acetylene. In separate experiments, contact with Catalyst B, Catalyst C, Catalyst D, Catalyst E, Catalyst F, Catalyst G, Catalyst H, and Catalyst I under the same conditions selectively hydrogenates an ethylene feedstream containing about 1% acetylene. After the selective hydrogenations, Catalysts A, B, C, D, E, F, G, H, and I are separately regenerated through the steam/air regeneration process.

Catalysts A, B, D, E, F, G, H, and I retain a greater percentage of their activity after regeneration than Catalyst C. The presence of the inorganic salts selected from the group consisting of zirconium, one or more lanthanide, one or more alkaline earth, and mixtures thereof on the support in Catalysts A, B, D, E, F, G, H, and I is found to improve the activity of the selective hydrogenation catalyst after regeneration.

Catalyst C does not contain inorganic salts selected from the group consisting of zirconium, one or more lanthanide, one or more alkaline earth, and mixtures thereof on the support. Catalyst C is less regenerable than the catalysts that contain the inorganic salts on the support.

Multi-phase catalyst A has higher activity than single phase catalyst I that has the same composition. The formation of the multi-phase catalyst improves the activity over the activity of the single phase catalyst.

Other tests are performed with feedstreams of propylene, butylene, pentene, and hexene in place of the previously described ethylene feedstream. All of the feedstreams contain approximately 1% acetylene. The tests are run in the presence of hydrogen at a pressure of 10 bar at temperatures between approximately 45 and 120° C. The trends for Catalysts A through I with the various feedstreams are similar to the trends that were obtained with the ethylene feedstream.

The embodiments of the present invention may be embodied in other specific forms without departing from its essential characteristics. The described embodiments are to be considered in all respects only as illustrative and not as restrictive. The scope of the embodiments of the present invention is, therefore, indicated by the appended claims rather than by the foregoing description. All changes which come within the meaning and range of the equivalence of the claims are to be embraced within their scope.

What is claimed is:

1. A catalyst for selective hydrogenation of acetylene and dienes in a light olefin feedstream, comprising:
a first component selected from the group consisting of copper, gold, silver, and mixtures thereof;
a second component selected from the group consisting of nickel, platinum, palladium, iron, cobalt, ruthenium, rhodium, and mixtures thereof;
an inorganic support; and
at least one inorganic salt or oxide selected from the group consisting of zirconium, a lanthanide, an alkaline earth, and mixtures thereof.

2. The catalyst of claim 1, wherein said at least one inorganic salt or oxide is added to the support by impregnation, kneading, or milling.

3. The catalyst of claim 1, wherein the inorganic salt or oxide, the first component, the second component, and the support may be added in any order.

4. The catalyst of claim 1, wherein said catalyst comprises at least one fluorite.

5. The catalyst of claim 4, wherein said fluorite is formed after calcination, use, or regeneration of said catalyst.

6. The catalyst of claim 1, wherein said first component comprises palladium and said second component comprises silver.

7. The catalyst of claim 1, wherein said inorganic salt is selected from the group consisting of nitrates, acetates, chlorides, carbonates, and mixtures thereof.

8. The catalyst of claim 1, wherein a weight percent of said inorganic salt or oxide is in the range of approximately 0.01% to approximately 50% by weight.

9. The catalyst of claim 1, wherein said catalyst is a multi-phase catalyst.

10. The catalyst of claim 9, wherein said catalyst is prepared with a water solution of at least two water-soluble salts selected from the group consisting of copper, gold, silver, nickel, platinum, palladium, iron, cobalt, ruthenium, rhodium, zirconium, a lanthanide, an alkaline earth, and mixtures thereof.

11. A process for selectively hydrogenating acetylene and dienes in a light olefin feedstream, comprising contacting said feedstream with hydrogen in the presence of a catalyst comprising a first component selected from the group consisting of copper, gold, silver and mixtures thereof, a second component selected from the group consisting of nickel, platinum, palladium, iron, cobalt, ruthenium, rhodium, and mixtures thereof, an inorganic support, and at least one inorganic salt or oxide selected from the group consisting of zirconium, a lanthanide, and an alkaline earth.

12. The process of claim 11, wherein said light olefin feedstream comprises at least one olefin having a carbon number between C₂ through C₆.

13. The process of claim 11, wherein said light olefin feedstream comprises at least one olefin selected from the group consisting of ethylene, propylene, butylene, pentene, and hexene.

14. The process of claim 11, wherein said contacting is at a temperature between approximately 0° C. and approximately 250° C.

15. The process of claim 11, wherein said contacting is at a pressure of approximately 0.01 bar to approximately 50 bar.

16. The process of claim 11, wherein said catalyst comprises at least one fluorite.

17. The process of claim 11, wherein said catalyst is a multi-phase catalyst.

18. The process of claim 11, wherein said first component comprises silver and said second component comprises palladium.

19. The process of claim 11, wherein said light olefin feedstream is an ethylene feedstream.

20. A method of preparing a multi-phase catalyst for selective hydrogenation of acetylene and diene in a light olefin feedstream, comprising:
forming a single aqueous solution of at least two water-soluble salts selected from the group consisting of copper, gold, silver, nickel, platinum, palladium, iron, cobalt, ruthenium, rhodium zirconium, a lanthanide, an alkaline earth, and mixtures thereof;
contacting said single aqueous solution with an inorganic support selected from the group consisting of silica and alumina; and
calcining said inorganic support and said single aqueous solution under a condition to form said multi-phase catalyst, wherein said multi-phase catalyst comprises at least one inorganic salt or oxide selected from the group consisting of zirconium, a lanthanide, and an alkaline earth.

21. The method of claim 20, further comprising removing the water from said single aqueous solution before calcining.

22. The method of claim 21, wherein removing the water comprises drying said single aqueous solution.

23. The method of claim 20, wherein said inorganic support is silica or alumina.

24. The method of claim 20, wherein said at least two water-soluble salts are salts selected from the group consisting of nitrates, acetates, oxalates, hydroxides, and carbonates.
